(19) United States
(12) Patent Application Publication
(10) Pub. No.: US 2010/0318102 A1
(43) Pub. Date: Dec. 16, 2010

(54) Needle holder with integrated scissors for microsurgery

(75) Inventor: Ming-Huei Cheng, Taipei City (TW)

Correspondence Address: BACON & THOMAS, PLLC, 625 SLATERS LANE, FOURTH FLOOR, ALEXANDRIA, VA, 22314-1176, US

(73) Assignees: Ming-Huei CHENG, Taipei City (TW); Steve L. HENRY, Taipei City (TW); Holger ENGEL, Taipei City (TW)

(21) Appl. No.: 12/801,413

(22) Filed: Jun. 8, 2010

(30) Foreign Application Priority Data
Jun. 15, 2009 (TW) 098210648

(51) Int. Cl. A61B 17/10

(52) U.S. Cl. 606/139

(57) ABSTRACT

A needle holder with integrated scissors for microsurgery is disclosed, which comprises a pair of elongated arms, and a pivot means. Each of the elongated arms includes: a clamping part disposed at a first end of the elongated arm, and having a clamping surface; a holding part disposed at a second end of the elongated arm; a hole disposed at a location intermediate the clamping part and the holding part; and a scissor blade disposed at a location intermediate the hole and the clamping part. In addition, the clamping surfaces correspond to each other, and the scissor blades are arranged to be in an overlapping cutting relation when the clamping surfaces are in a closed position. Further, the pivot means is disposed in the holes for pivoting the elongated arms.

### BACKGROUND OF THE INVENTION

1. Field of the Invention

The present invention relates to a needle holder with integrated scissors for microsurgery and, more particularly, to a needle holder with scissors for microsurgery, which is able to cut the suturing thread to improve the suturing efficiency during the microsurgery operation.

2. Description of Related Art

In a typical use common suturing procedure, various surgical instruments, such as needle holders, forceps, and scissors, are needed. The surgeon must hold forceps in one hand and a needle holder in the other hand to suture wounds. The needle holder is used to clamp a curved needle, and a suture thread is bound to the curved needle. After wound edges are closed by the suture thread and the thread is then tied into a knot by a surgeon, the surgeon exchanges the needle holder with the scissor to cut the thread. When there are several or large wounds going to be closed, it is necessary for the surgeon to pick up the needle holder and the scissor and place them back on the instrument tray multiple times. The continual picking up and putting down of surgical instruments is time-consuming, which may cause the surgeon distraction and consume the patient's physical vigor. In particular, when the microsurgery is performed, continual picking up and putting down of surgical instruments also decreases the operational efficiency.

FIG. 1 is perspective view of a conventional needle holder for microsurgery, which comprises: a pair of elongated arms 10, and a pivot means 11. Each of the elongated arms 10 comprises: a clamping part 101 disposed at a first end of the elongated arm 10, and having a clamping surface 1011; a holding part 102 disposed at a second end of the elongated arm 10; and a hole 103 disposed at a location intermediate the clamping part 101 and the holding part 102. The pivot means 11 is disposed in the holes 103 for pivoting the elongated arms 10 together.

The clamping parts 101 of the needle holder clamp a curved needle to close the wound edge and knot the suture thread. Then, the needle holder is put down and scissors are picked up for microsurgery to cut the thread. Because the microsurgery is very delicate, the holding parts 102 of the needle holder must be designed into plates to facilitate the microsurgery operation. Although the needle holder with plate-shaped holding parts 102 can be effectively applied on microsurgery, scissors for microsurgery must be used to cut the thread after the suture process. Hence, some operation time is still wasted during picking up and putting down of the surgical instruments. Therefore, it is desirable to provide a needle holder with integrated scissors for microsurgery, to eliminate the steps for the exchange of the needle holder and the scissor; thereby the surgical efficiency can be improved.

### SUMMARY OF THE INVENTION

The object of the present invention is to provide a needle holder with integrated scissors for microsurgery, which has a bi-function of holding a needle and cutting a suture thread to improve the operation efficiency of the microsurgery.

To achieve the object, the needle holder with integrated scissors for microsurgery comprises: a pair of elongated arms, and a pivot means. Herein, each of the elongated arms comprises: a clamping part disposed at a first end of the elongated arm, and having a clamping surface; a holding part disposed at a second end of the elongated arm; a hole disposed at a location intermediate the clamping part and the holding part; and a scissor blade disposed at a location intermediate the hole and the clamping part. In addition, the clamping surfaces correspond to each other, and the scissor blades are arranged to be in an overlapping cutting relation when the clamping surfaces are in a closed position. Further, the pivot means is disposed in the holes for pivoting the elongated arms together.

The needle holder with integrated scissors for microsurgery of the present invention is a multi-functional needle holder integrated with scissors, which can achieve the purposes of holding a needle and cutting a suture thread at the same time. Hence, the surgeon can suture the wound, knot the suture thread and cut the thread by using only one surgical instrument, i.e. the needle holder with integrated scissors for microsurgery of the present invention, so that the surgeon does not have to keep picking up and putting down the needle holder and the scissor multiple times. Because the procedure of exchanging the needle holder and the scissor can be eliminated by using the needle holder with integrated scissors for microsurgery of the present invention, the operation time can be reduced. Therefore, the consumption of the physical vigor to the surgeon and the patient can be decreased, and the treatment cost can also be decreased.

According to the needle holder with integrated scissors for microsurgery of the present invention, one holding part has a punch, another holding part has a protrusion part, and the protrusion part is mounted into the punch and movable in the punch. In addition, the holding parts are plates, preferably, in order to reduce the volume of the needle holder. For the delicate microsurgery, the plate-shaped holding parts can improve the handiness of the operation.

In addition, according to the needle holder with integrated scissors for microsurgery of the present invention, each of the holding parts may further comprise an anti-slip part. Besides, each of the holding parts may also further comprise a fixing part. Preferably, each of the holding parts further comprises an anti-slip part and a fixing part, and the anti-slip part and the fixing part are respectively disposed in two sides of the elongated arm. The anti-slip parts can prevent the dropping of the needle holder during surgery, and the fixing part can maintain the clamping parts in a closed position to facilitate the performance of operation.

Furthermore, according to the needle holder with integrated scissors for microsurgery of the present invention, a length ratio in a longitude direction between the clamping part and the scissor blade may be in a range of 1:3 to 3:1. Preferably, the length ratio in a longitude direction between the clamping part and the scissor blade is 1:1.

According to the needle holder with integrated scissors for microsurgery of the present invention, the clamping surfaces of the clamping parts may be roughened surfaces, which can increase the friction between the clamping surfaces and the curved needle; thereby the risk of the needle being dropped during the operation can be decreased. In addition, the cutting surfaces of the scissor blades can have serrated edges, or straight edges. Preferably, at least one cutting surface of the scissor blades has a serrated edge, in order to increase the friction between the cutting surface and the suture thread. Hence, the relative position between the suture thread and the scissor blades can be fixed, so it is possible for the scissor blades to precisely cut the thread at a determined position.

### BRIEF DESCRIPTION OF THE DRAWINGS

FIG. 1 is a perspective view of a conventional needle holder for microsurgery;

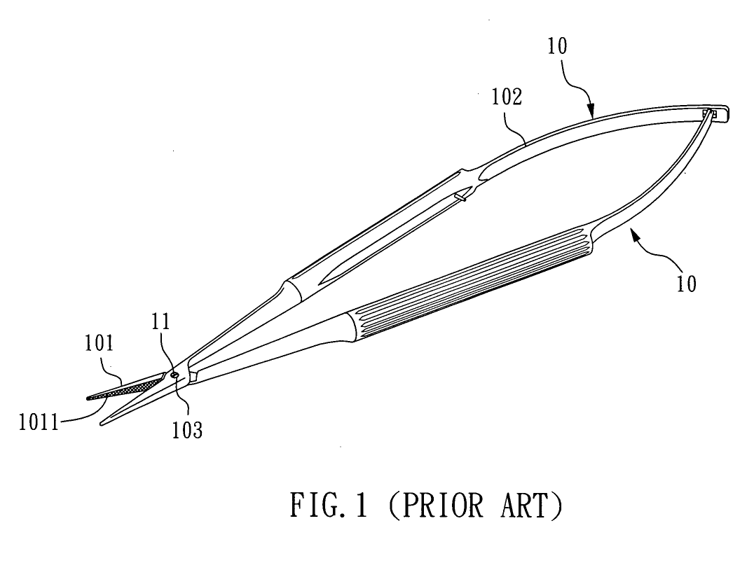

FIG. 2 is a perspective view of a needle holder with integrated scissors in a preferred embodiment of the present invention;

FIG. 3 is a perspective view of the first ends of the elongated arms in a preferred embodiment of the present invention; and

FIG. 4 is a perspective view of the second ends of the elongated arms in a preferred embodiment of the present invention.

### DETAILED DESCRIPTION OF THE PREFERRED EMBODIMENT

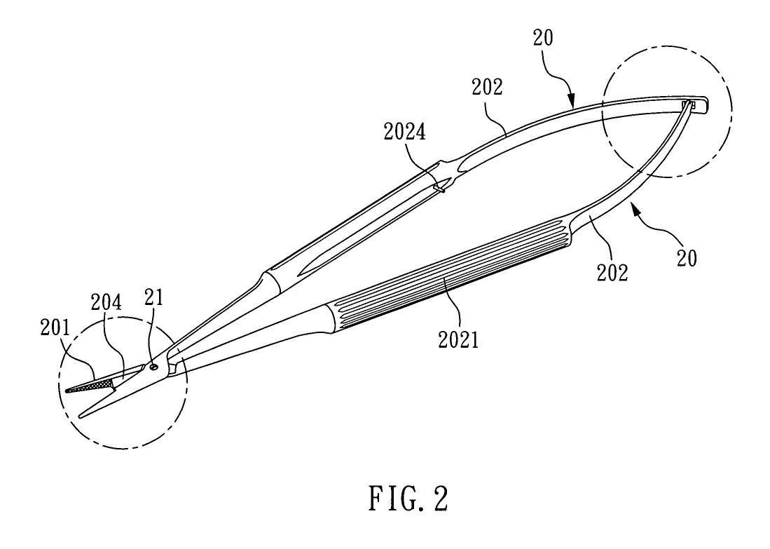

FIG. 2 is a perspective view of a needle holder with integrated scissors in a preferred embodiment of the present invention.

As shown in FIG. 2, the needle holder with integrated scissors for microsurgery of the present embodiment comprises: a pair of elongated arms 20, and a pivot means 21. Herein, each of the elongated arms 20 comprises: a clamping part 201 disposed at a first end of the elongated arm 20; a holding part 202 disposed at a second end of the elongated arm 20; a hole (not shown in the figure) disposed at a location intermediate the clamping part 201 and the holding part 202; and a scissor blade 204 disposed at a location intermediate the hole (not shown in the figure) and the clamping part 201. In addition, the pivot means 21 is disposed in the holes (not shown in the figure) for pivoting the elongated arms 20 together.

In order to prevent the needle holder from being dropped during the operation, the holding parts 202 further comprise anti-slip parts 2021. In addition, the holding parts 202 further comprise fixing parts 2024, and the fixing parts 2024 and the anti-slip parts 2021 are respectively disposed in two sides of the elongated arms 20. The fixing parts 2024 can maintain the two clamping parts 201 in a closed position to facilitate the surgeon to handle the needle holder. Furthermore, the needle holder with integrated scissors can be made from tungsten steel, titanium, or stainless steel. In the present embodiment, the needle holder with integrated scissors is made from stainless steel.

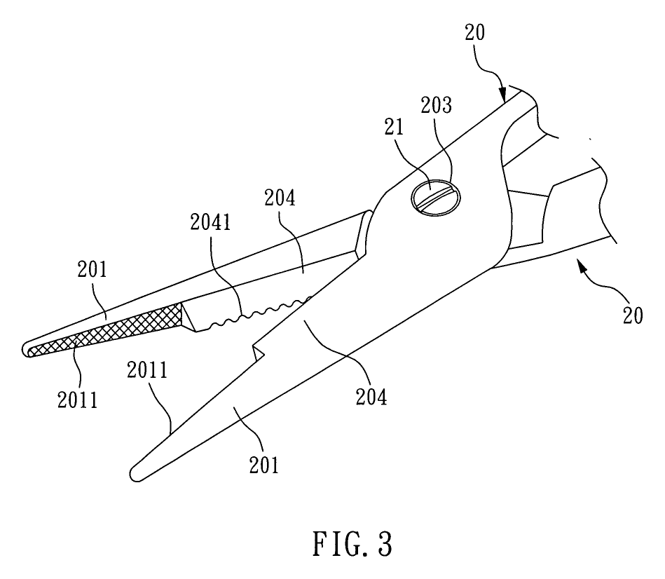

FIG. 3 is a perspective view of the first ends of the elongated arms in the present embodiment. The relative position between the clamping parts, the scissor blades, and the holes can be clearly understood by viewing FIG. 3.

As shown in FIG. 3, the pivot means 21 is disposed in the holes 203 for pivoting the elongated arms 20 together. In addition, the clamping parts 21 have clamping surfaces 2011, and the two clamping surfaces 2011 correspond to each other. The clamping surfaces 2011 of the clamping parts are roughened surfaces, in order to increase the friction between the clamping surfaces 2011 and the curved needle (not shown in the figure) to prevent the curved needle from being dropped. Furthermore, the scissor blades 204 are disposed at a location intermediate the holes 203 and the clamping parts 201, and the cutting surface 2041 of one scissor blade 204 has a serrated edge, in order to increase the friction between the cutting surface 2041 and the suturing thread (not shown in the figure) to facilitate the thread cutting. When the clamping surfaces 2011 of the clamping parts 201 are in a closed position, the scissor blades 204 are arranged to be in an overlapping cutting relation to cut the suturing thread. In addition, a length ratio in a longitude direction between the clamping part 201 and the scissor blade 204 can be in a range of 1:3 to 3:1. In the present embodiment, the length ration in the longitude direction between the clamping part 201 and the scissor blade 204 is 1:1.

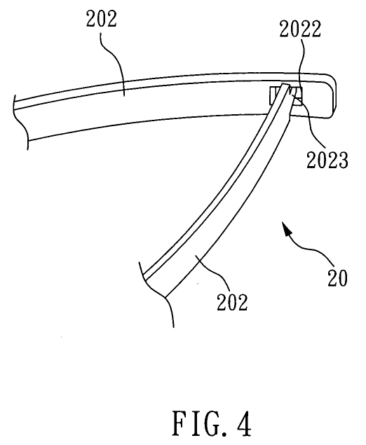

FIG. 4 is a perspective view of the second ends of the elongated arms in the present embodiment. The structure of the holding parts can be clearly understood by FIG. 4.

As shown in FIG. 4, the holding parts 202 are plates, which means that the holding parts 202 are configured from metal sheets. In the structure of the plate-shaped holding parts 202, one holding part 202 has a punch 2022, the other holding part 202 has a protrusion part 2023, and the protrusion part 2023 is mounted into the punch 2022 and is movable in the punch 2022. Referring to FIGS. 2 and 4, according to the relation position between the punch 2022 and the protrusion part 2023 of the holding parts 202, the clamping parts 201 and the scissor blades 204 are in a closed position when the holding parts 202 are pressed. In addition, when the pressure applied on the holding parts 202 is released, the restorative force of the holding parts 202 configured by plates can return the clamping parts 201 or the scissor blades 204 to an open position. Furthermore, the holding parts 202 are configured by plates, so the needle holder can be lighter and handier and more convenient in the microsurgery.

In conclusion, the needle holder with integrated scissors for microsurgery is a multi-functional needle holder, which can hold a curved needle and cut a suturing thread. Hence, the surgeon does not have to exchange the needle holder and the scissors during the operation, and the operation efficiency can be improved. In addition, the conventional holding parts of the needle holder are too bulky and heavy, and may influence the operation handiness in microsurgery. On the contrary, the holding parts of the needle holder with integrated scissors for microsurgery of the present invention is designed as plates, and the operation handiness can be maintained by using the needle holder of the present invention. Furthermore, according to the needle holder with integrated scissors for microsurgery of the present invention, at least one scissor blade further comprises a serrated edge, which can increase the friction between the scissor blade and the suturing thread to keep the thread in a precise cutting position.

Although the present invention has been explained in relation to its preferred embodiment, it is to be understood that many other possible modifications and variations can be made without departing from the scope of the invention as hereinafter claimed.

What is claimed is:

1. A needle holder with integrated scissors for microsurgery, comprising:
a pair of elongated arms, each of the elongated arms comprising:
a clamping part disposed at a first end of the elongated arm, and having a clamping surface;
a holding part disposed at a second end of the elongated arm;
a hole disposed at a location intermediate the clamping part and the holding part; and
a scissor blade disposed at a location intermediate the hole and the clamping part,
wherein, the clamping surfaces correspond to each other, and the scissor blades are arranged to be in an overlapping cutting relation when the clamping surfaces are in a closed position, and
a pivot means disposed in the holes for pivoting the elongated arms together.

2. The needle holder with integrated scissors for microsurgery as claimed in claim 1, wherein one holding part has a punch, another holding part has a protrusion part, and the protrusion part is mounted into the punch and is movable in the punch.

3. The needle holder with integrated scissors for microsurgery as claimed in claim 1, wherein the clamping surfaces of the clamping parts are roughened surfaces.

4. The needle holder with integrated scissors for microsurgery as claimed in claim 1, wherein the holding parts are plates.

5. The needle holder with integrated scissors for microsurgery as claimed in claim 1, wherein at least one cutting surface of the scissor blades has a serrated edge.

6. The needle holder with integrated scissors for microsurgery as claimed in claim 1, wherein the cutting surfaces of the scissor blades have straight edges.

7. The needle holder with integrated scissors for microsurgery as claimed in claim 1, wherein each of the holding parts further comprises an anti-slip part.

8. The needle holder with integrated scissors for microsurgery as claimed in claim 1, wherein each of the holding parts further comprises a fixing part.

9. The needle holder with integrated scissors for microsurgery as claimed in claim 1, wherein each of the holding parts further comprises an anti-slip part and a fixing part, and the anti-slip part and the fixing part are respectively disposed in two sides of the elongated arm.

10. The needle holder with integrated scissors for microsurgery as claimed in claim 1, wherein a length ratio in a longitude direction between the clamping part and the scissor blade is in a range of 1:3 to 3:1.
